(19) United States
(12) Patent Application Publication
(10) Pub. No.: US 2010/0291027 A1
(43) Pub. Date: Nov. 18, 2010

(54) HYALURONIC ACID (HA) INJECTION VEHICLE

(76) Inventor: Jason Campbell, Helena, AL (US)

Correspondence Address: Ballard Spahr LLP, SUITE 1000, 999 PEACHTREE STREET, ATLANTA, GA, 30309-3915, US

(21) Appl. No.: 12/779,138

(22) Filed: May 13, 2010

Related U.S. Application Data
(60) Provisional application No. 61/178,271, filed on May 14, 2009.

(51) Int. Cl. A61K 38/20; A61K 38/02; A61K 38/43; A61K 39/395; A61K 31/7088; A61P 1/04; A61P 11/06; A61P 1/12; A61P 7/02; A61P 25/18; A61P 31/04; A61P 35/00; A61P 3/04; A61P 11/08; A61P 3/10; A61P 25/20; A61P 3/02; A61P 5/14; A61K 38/18; A61K 31/343; A61K 31/137; A61K 31/485; A61K 31/616; A61K 33/08; A61K 38/21; A61K 39/00; A61P 5/30; A61P 25/16; A61P 9/12; A61K 31/43; A61K 31/65; A61K 31/7048; A61K 31/661; A61K 31/56; A61K 38/27; A61K 38/28; A61K 38/48; A61K 38/19; A61K 39/35

(52) U.S. Cl. 424/85.2; 514/1.1; 424/94.1; 424/130.1; 514/44 R; 514/44 A; 514/8.1; 514/463; 514/653; 514/282; 514/165; 424/690; 424/85.4; 424/184.1; 514/210.05; 514/152; 514/31; 514/137; 514/169; 514/11.3; 514/5.9; 424/94.64; 424/141.1; 424/85.1; 424/275.1

(57) ABSTRACT

Disclosed herein are compositions that exhibit viscosities suitable for injectable formulations. The compositions comprise bioactive agent-loaded microparticles and hyaluronic acid or a salt thereof in a suitable liquid pharmaceutical carrier. Also disclosed are methods of making and using the compositions.

### CROSS-REFERENCE TO RELATED APPLICATIONS

This application claims the benefit of priority to U.S. Provisional Application No. 61/178,271, filed May 14, 2009, the entire contents of which are incorporated herein by reference.

### BACKGROUND

It is often desirable to deliver a bioactive agent to a subject through a controlled release composition, such as one comprising a bioactive agent-loaded microparticle, by injecting the composition into a fluid or tissue of a subject. Such an administration mode can be limited by the amount of the composition that can readily suspended and injected through a syringe, particularly when the formulation comprises microparticles. Syringe clogging and poor suspendability are both problems encountered with injectable microparticle compositions. Various efforts, including adding excipients to the compositions, have been used to overcome such problems, many of which have not resulted in optimal suspendability or injectability. A need therefore exists for improved compositions and methods that address these problems.

### SUMMARY

Disclosed herein are compositions that exhibit viscosities suitable for injectable formulations. The compositions of the invention comprise a liquid pharmaceutical carrier having dissolved or dispersed therein (a) hyaluronic acid or salt thereof at a concentration of from about 0.01% to about 5% by weight; and (b) bioactive agent-loaded biodegradable microparticles.

Also disclosed are methods for preparing the compositions, comprising (a) preparing a formulation by admixing into a liquid pharmaceutical carrier (i) hyaluronic acid or a salt thereof to a concentration of from about 0.01% to about 5% by weight of the liquid pharmaceutical carrier; and (ii) bioactive agent-loaded biodegradable microparticles; (b) autoclaving the formulation for a time ranging from about 15 to about 30 minutes at a temperature of from about 110° C. to about 130° C.; and (c) cooling the formulation to room temperature.

Also disclosed are methods for administering a bioactive agent to a subject, comprising: (a) injecting into a subject a composition comprising a liquid pharmaceutical carrier having dissolved or dispersed therein (i) hyaluronic acid or salt thereof at a concentration of from about 0.01% to about 5% by weight; and (ii) bioactive agent-loaded biodegradable microparticles; and (b) allowing the biodegradable microparticle to degrade, thereby delivering the bioactive agent to the subject.

### DETAILED DESCRIPTION

The present invention relates generally to injectable pharmaceutical compositions comprising hyaluronic acid or a salt thereof and bioactive agent-loaded biodegradable microparticles. Methods of preparing and using the compositions are also provided.

“Hyaluronic acid” or a “salt of hyaluronic acid” (also called hyaluronan or hyaluronate) refers to a non-sulfated polysaccharide which is a glycosaminoglycan. Hyaluronic acid comprises an alternating copoly(β-D-glucuronic acid-β-D-N-acetyl-glucosamin) or a salt thereof. Salts of the hyaluronic acid include potassium and sodium salts, among others.

The term “microparticle” is used herein to refer generally to a variety of structures having sizes from about 10 nm to 2000 microns (2 millimeters) and includes microcapsules, microspheres, nanoparticles, nanocapsules, nanospheres as well as particles, in general, that are less than about 2000 microns (2 millimeters).

“Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. Bioactive agents also include those substances which affect the structure or function of a subject, or a pro-drug, which becomes bioactive or more bioactive after it has been placed in a predetermined physiological environment.

“Autoclaving,” as used herein, refers to a process in which the compositions of the invention are exposed to high-pressure steam at an elevated temperature. A variety of commercially available autoclaves can be used.

The hyaluronic acid or salt thereof is present in the compositions of the invention at a concentration of from about 0.01% to about 5% by weight, from about 0.1% to about 2%, or from 0.1 to about 1% by weight. For example, the hyaluronic acid or salt thereof can be present at a concentration of 0.2%, 0.5%, 0.6%, 0.65%, 0.7%, 0.75%, or 1% by weight. In one example, the hyaluronic acid or salt thereof can be present at a concentration of from about 0.8% to about 1.2%.

The hyaluronic acid or salt thereof preferably has a molecular weight of from about 0.5×10⁵ to about 10×10⁶ Daltons. In a further aspect, the hyaluronic acid or salt thereof has a molecular weight of from about 5.2×10⁵ to 1.46×10⁶ Daltons. For example, the hyaluronic acid or salt thereof can have a molecular weight of about 5.2×10⁵ Daltons, about 8.0×10⁵ Daltons, or about 1.46×10⁶ Daltons.

The hyaluronic acid or salt thereof can be obtained commercially or prepared or extracted from natural sources using methods known in the art. As an example, Sodium hyaluronate is sold as a solution or dispersion under the tradename HEALON. HEALON comprises 10 mg/mL of sodium hyaluronate dissolved in physiological sodium chloride phosphate buffer pH 7.0-7.51. Lyophilized hyaluronic acid powders can also be obtained commercially in a range of molecular weights.

The liquid pharmaceutical carrier can be any suitable carrier known in the pharmaceutical arts. Examples of liquid carriers are sugar syrup, peanut oil, olive oil, and water. The liquid carrier is preferably aqueous, and can comprise one or more salts, such as saline or phosphate buffered saline. The liquid pharmaceutical carrier is preferably kept at a physiological acceptable pH. Other pharmaceutically acceptable carriers or components that can be mixed with the bioactive agent can include, for example, a fatty acid, a sugar, a salt, a water-soluble polymer such as polyethylene glycol, a protein, polysacharride, or carboxymethyl cellulose, a surfactant, a plasticizer, a high- or low-molecular-weight porosigen such as polymer or a salt or sugar, or a hydrophobic low-molecular-weight compound such as cholesterol or a wax.

The composition can optionally comprise a surfactant or other additive used in pharmaceutical formulations. In one aspect, the composition comprises polyoxyethylene sorbitan monolaurate. The polyoxyethylene sorbitan monolaurate can comprise polysorbate 20, 40, 60, or 80, commonly sold under the tradenames TWEEN 20, TWEEN, 40, TWEEN 60, and TWEEN 80. The polyoxyethylene sorbitan monolaurate is preferably polyoxyethylene (20) sorbitan monolaurate polyoxyethylene, which refers to the total number of oxyethylene —(CH₂CH₂O)— groups found in the polymer.

The microparticles in the compositions can be present in any suitable weight percent, for example up to 1000 mg/mL of the liquid carried. The microparticles generally comprise a biodegradable polymeric microparticle matrix which has encapsulated therein the bioactive agent. Such microparticles can be prepared using methods known in the art. Preferably, the microparticles are prepared using an emulsion or double-emulsion technique known in the art. The polymer can be one or more of polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones.

The microparticle can also comprise a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof. The polymers can also comprise polyethylene glycol (PEG), polyethylene oxide (PEO), or a combination of such with one or more other disclosed polymers.

Any lactide residue can be used, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof.

Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When the biodegradable polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

The polymer can also be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

In one aspect, the polymer can comprise a terpolymer. The terpolymers can be those terpolymers disclosed in U.S. patent application Ser. No. 12/269135, filed Nov. 12, 2008, (U.S. Patent Publication No. 2009/0124535) which is incorporated herein by this reference for all of its teachings of terpolymers.

Various forms of the bioactive agent can be used, which are capable of being released from the compositions into adjacent tissues or fluids of a subject. A liquid or solid bioactive agent can be incorporated into the compositions described herein. The bioactive agents are at least very slightly water soluble, and preferably moderately water soluble. The bioactive agents can include salts of the active ingredient. As such, the bioactive agents can be acidic, basic, or amphoteric salts. They can be nonionic molecules, polar molecules, or molecular complexes capable of hydrogen bonding. The bioactive agent can be included in the compositions in the form of, for example, an uncharged molecule, a molecular complex, a salt, an ether, an ester, an amide, polymer drug conjugate, or other form to provide the effective biological or physiological activity.

Examples of bioactive agents that incorporated into the compositions include, but are not limited to, peptides, proteins such as hormones, enzymes, antibodies, antibody fragments, and the like, nucleic acids such as aptamers, iRNA, DNA, RNA, antisense nucleic acid or the like, antisense nucleic acid analogs or the like, low-molecular weight compounds, or high-molecular-weight compounds. Bioactive agents contemplated for use in the disclosed micropartices include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Other bioactive agents include androgen inhibitors, polysaccharides, growth factors (e.g., a vascular endothelial growth factor—VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents in the compositions include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

The composition can comprise a large number of bioactive agents either singly or in combination. Other bioactive agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenytoin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, cox sackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, or a combination thereof. In one aspect, the bioactive agent can be a combination of Rifampicin (Rifampin in U.S.) and Minocycline.

The compositions of the invention can be prepared by (a) preparing a formulation by admixing into the liquid pharmaceutical carrier (i) hyaluronic acid or a salt thereof to a concentration of from about 0.01% to about 5% by weight of the liquid pharmaceutical carrier; and (ii) bioactive agent-loaded biodegradable microparticles at a concentration of up to 1000 mg/mL by weight of the liquid pharmaceutical carrier; (b) autoclaving the formulation for a time ranging from about 15 to about 30 minutes at a temperature of from about 110° C. to about 130° C.; and (c) cooling the formulation to room temperature.

The admixing step (a) can be carried using conventional techniques. In some aspects, it can be desirable to shake the formulation admixed together in step (a) for an extended period of time, such as several minutes, several hours, or even overnight. The autoclaving step (b) is carried out by sealing the formulation in a suitable vessel followed by autoclaving the formulation. The cooling step (c) can be carried out by simply allowing the formulation to cool, or cooling the formulation by refrigeration or another suitable means.

The compositions can be administered to a subject to deliver to the subject a bioactive agent, through the degradation of the biodegradable microparticle. The method for administration comprises: (a) injecting into a subject a composition comprising a liquid pharmaceutical carrier having dissolved or dispersed therein (i) hyaluronic acid or salt thereof at a concentration of from about 0.01% to about 5% by weight; and (ii) bioactive agent-loaded biodegradable microparticles at a concentration of up to 1000 mg/mL; and (b) allowing the biodegradable microparticle to degrade, thereby delivering the bioactive agent to the subject.

Due to the satisfactory viscosity of the compositions of the inventions, the compositions can be drawn into a syringe by a user and held in the syringe for as much as 5 minutes prior to performing the injection.

The compositions of the invention can be administered to any desired subject. The subject can be a vertebrate, such as a mammal, a fish, a bird, a reptile, or an amphibian. The subject of the herein disclosed methods can be, for example, a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

What is claimed is:

1. A composition comprising a liquid pharmaceutical carrier having dissolved or dispersed therein (a) hyaluronic acid or salt thereof at a concentration of from about 0.01% to about 5% by weight; and (b) bioactive agent-loaded biodegradable microparticles.

2. The composition of claim 1 wherein the hyaluronic acid or salt thereof is present at a concentration of from about 0.1 to about 1% by weight.

3. The composition of claim 1 wherein the hyaluronic acid or salt thereof has a molecular weight of from about 0.5×10⁵ to about 10×10⁶ Daltons.

4. The composition of claim 1 wherein the liquid pharmaceutical carrier comprises a physiological buffer.

5. The composition of claim 1 wherein the liquid pharmaceutical carrier comprises saline.

6. The composition of claim 1 further comprising polyoxyethylene sorbitan monolaurate at a concentration of from about 0.01% to about 1% by weight.

7. The composition of claim 6 wherein the polyoxyethylene sorbitan monolaurate is at a concentration of from about 0.1% to about 1% by weight.

8. The composition of claim 6 wherein the polyoxyethylene sorbitan monolaurate is at a concentration of about 0.1%.

9. A method for preparing a pharmaceutical composition, comprising:
(a) preparing a formulation by admixing into a liquid pharmaceutical carrier (i) hyaluronic acid or a salt thereof to a concentration of from about 0.01% to about 5% by weight of the liquid pharmaceutical carrier; and (ii) bioactive agent-loaded biodegradable microparticles;
(b) autoclaving the formulation for a time ranging from about 15 to about 30 minutes at a temperature of from about 110° C. to about 130° C.; and
(c) cooling the formulation to room temperature.

10. The method of claim 9 wherein the hyaluronic acid or salt thereof is admixed into the liquid pharmaceutical carrier to a concentration of from about 0.1 to about 1% by weight.

11. The method of claim 9 wherein the hyaluronic acid or salt thereof has a molecular weight of from about 0.5×10⁵ to about 10×10⁶ Daltons.

12. The method of claim 9 wherein the liquid pharmaceutical carrier comprises a physiological buffer.

13. The method of claim 9 wherein the liquid pharmaceutical carrier comprises saline.

14. The method of claim 9 further comprising admixing into the liquid pharmaceutical carrier, prior to step (b), polyoxyethylene sorbitan monolaurate to a concentration of from about 0.01% to about 1% by weight.

15. A method for administering a bioactive agent to a subject, comprising:
(a) injecting into a subject a composition comprising a liquid pharmaceutical carrier having dissolved or dispersed therein (i) hyaluronic acid or salt thereof at a concentration of from about 0.01% to about 5% by weight; and (ii) bioactive agent-loaded biodegradable microparticles;
(b) allowing the biodegradable microparticle to degrade, thereby delivering the bioactive agent to the subject.

16. The method of claim 15 wherein prior to injecting the composition, the composition is drawn into a syringe and held in the syringe for a time of up to 5 minutes.

17. The method of claim 15 wherein the hyaluronic acid or salt thereof is present at a concentration of from about 0.1 to about 1% by weight.

18. The method of claim 15 wherein the hyaluronic acid or salt thereof has a molecular weight of from about 0.5×10⁵ to about 10×10⁶ Daltons.

19. The method of claim 15 wherein the liquid pharmaceutical carrier comprises a physiological buffer.

20. The composition of claim 15 wherein the composition further comprises polyoxyethylene sorbitan monolaurate at a concentration of from about 0.01% to about 1% by weight.
